(19) United States
(12) Patent Application Publication
(10) Pub. No.: US 2013/0041446 A1
(43) Pub. Date: Feb. 14, 2013

(54) SELECTIVE NERVE STIMULATION FOR RELIEF OF ABDOMINAL PAIN

(71) Applicant: Charles Henry Knowles, London (GB)

(72) Inventor: Charles Henry Knowles, London (GB)

(73) Assignee: Queen Mary & Westfield College, University of London, Greater London (GB)

(21) Appl. No.: 13/569,441

(22) Filed: Aug. 8, 2012

(30) Foreign Application Priority Data
Aug. 8, 2011 (GB) 1113602.5

(51) Int. Cl. A61N 1/05

(52) U.S. Cl. 607/118

(57) ABSTRACT

The present invention provides a method of abdominal pain relief in a subject comprising the steps of: identifying sub-diaphragmatic vagus nerve branches; applying an electrode to said nerve branch; stimulating the subject's vagus nerve branch by the application of electrical pulses through said electrode to said nerve in a given pattern and at a rate controlled by a controller such as to relieve pain. The electrical pulse may be applied at a number of frequencies, for a given duration with a given rest period and may be of a given magnitude, to control the pain experienced by the patient. In an example arrangement the stimulation is applied to the vagus branch of the celiac branch or posterior vagal trunk of the sub-diaphragmatic vagus nerve.

### CROSS-REFERENCE TO RELATED APPLICATIONS

This application claims the benefit of United Kingdom Application No. 1113602.5 filed on Aug. 08, 2011, which is incorporated herein by reference in its entirety.

### TECHNICAL FIELD

This document relates to neural stimulation methods and systems. For example, the described methods and systems relate to neural stimulation of sub-diaphragmatic nerves for the relief of abdominal pain.

### BACKGROUND

The gastrointestinal (GI) tract is a system of organs within multicellular animals that facilitate the ingestion, digestion and absorption of food with subsequent defecation of waste. A complex arrangement of nerves and ancillary cells contribute to the sensorimotor apparatus required to subserve such essential functions that are, with the exception of the extreme upper and lower ends of the GI tract, normally subconscious. However they also have the potential to provide conscious awareness of injury. Although this function can be protective, when dysregulated, particularly on a chronic basis, the same system can lead to considerable morbidity.

Abdominal pain is the commonest cause of presentation to a surgeon or gastroenterologist (Shaheen et al., 2006), with abdominal or pelvic viscera commonly implicated, or proven to be the site of origin. Acute abdominal pain may be caused by several mechanisms with clinical presentation commonly reflecting the predominant underlying aetiology. Broadly, pain may arise as a result of visceral stretching as occurs with obstruction, inflammation as occurs in inflammatory bowel disease, or invasion/compression of nerves such as might occur in some cases of cancer. However, acute pain e.g. trauma/surgery, or that with a treatable cause e.g. inflammation, are often less problematic than chronic pain, particularly when this is unexplained.

The current mainstay of management for patients with chronic visceral pain and frequent accompanying dysmotility is supportive with attention to nutrition, pain management and psychological wellbeing. Definitive treatments are sadly lacking and there is thus a need to develop new approaches to therapy. Neuromodulation is one such possibility but it has yet to be successfully applied to abdominal pain relief. The concept of using neurostimulation for visceral problems is not new and the technique is well known for use through spinal cord stimulation for abdominal & visceral pain relief and peripheral stimulation for gastrointestinal sensrimotor dysfunction. Peripheral neuromodulation has expanded the treatment of certain upper and lower gastrointestinal conditions with sacral nerve stimulation (SNS) (Interstim®, Medtronic Inc., Minneapolis, Minn.) becoming the first line surgical treatment of faecal incontinence (Wexner et al., 2010) and an evolving management option for intractable constipation (Knowles et al., 2009; Kamm et al., 2010; Knowles et al., 2011). Gastric electrical stimulation (GES) (Enterra®, Medtronic Inc. Minneapolis, Minn.) has acceptable results in the treatment of refractory idiopathic and diabetic gastroparesis (Abell et al., 2003), and may have some future value in the treatment of obesity (Champion et al., 2006). There is also one report of GES for intestinal pseudo-obstruction in which four patients had improvement in nausea and vomiting (Andersson et al., 2006).

No attempt has been made, however, to develop specific methods of GI neuromodulation tailored to abdominal pain relief. Although there are no direct data from humans, the effects of GES on nausea and vomiting (in gastroparesis) are now thought to be mediated at least in part by vagal afferent modulation (McCallum et al., 2010). Attempts are continuing to directly stimulate the small intestinal wall of dogs and pigs with the ultimate aim of inducing or reducing propulsive motility. Direct duodenal stimulation has been proposed as a treatment for obesity (Xu et al., 2011). The methodology includes sequential electrodes spaced directly on the bowel wall with rhythmical stimulation programs from proximal to distal—to induce peristalsis.

From the above, it will be appreciated that much work has been undertaken in the area of nerve stimulation for the purposes of modifying gastrointestinal function, but to date, little if any progress has been made in the area of abdominal pain relief.

### SUMMARY

Provided are systems and methods of abdominal pain relief in a subject comprising the steps of: identifying sub-diaphragmatic vagus nerve branches; applying an electrode to said nerve branch; stimulating the subject's vagus nerve branch by the application of electrical pulses through said electrode to said nerve in a given pattern and at a rate controlled by a controller such as to relieve pain.

Optionally, the portion of the vagus nerve that is identified is the celiac branch or sub-diaphragmatic posterior vagal trunk and the electrode is applied directly to said nerve. The electrical pulses optionally comprise a directional uniphasic current. Such a current is optionally applied periodically. When applied, such a current optionally comprises a current of between 0.25 mA and 2.0 mA. While a number of different frequencies are optionally used depending on the type of pain and intensity thereof, it has been found that frequencies on the range of between 5 Hz and 50 Hz are advantageous. For example, a frequency of 30 Hz is optionally used.

When applied, the stimulation is optionally applied at intervals for a given duration and the stimulation is optionally applied in an on/off ratio of between 1/10 and 1/1. The pulses are optionally applied for between 100 μs and 1000 μs, for example, for between 200 μs and 500 μs and, for example, for 500 μs.

Optionally, the stimulation is applied via an electrode having a plurality of electrode plates positioned along and alongside the nerve and electrical pulses are applied between different pairs thereof in accordance with a pre-defined protocol. The procedure itself optionally includes the attachment of the electrode to the identified nerve and optionally also includes the step of securing any electrical lead used for the supply of electricity to said electrode to the anterior wall of the stomach. The securing may be achieved by means of a slide coupling allowing the lead to slide within the coupling, thereby to accommodate movement.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagrammatic representation of the sub-diaphragmatic abdominal portion of a human body and illustrates the location of the nerves being used in an example method of the present application;

FIG. 2 illustrates where an implantable programmer used in an example method of the present application is optionally positioned relative to the liver and stomach;

FIG. 3 is a more detailed view of an electrode used in an example method of the present applicaiton and illustrates its location on the celiac nerve;

FIG. 4 is an illustration of the electrode shown in FIG. 3 and illustrates the physical connection such that it remains in the desired location;

FIG. 5 illustrates in partial cutaway from the exposed lesser omentum by schematic removal of the left lobe of the liver; and

FIG. 6 is a schematic representation of the type of electrode that may be used in an example method of the present applicaiton.

Like reference symbols in the various drawings indicate like elements.

### DETAILED DESCRIPTION

Provided are new methods and systems that are based on the observation that the sub-diaphragmatic vagus nerve (or main branch thereof) is a suitable target for stimulation such as to relieve and possibly eliminate abdominal pain in patients.

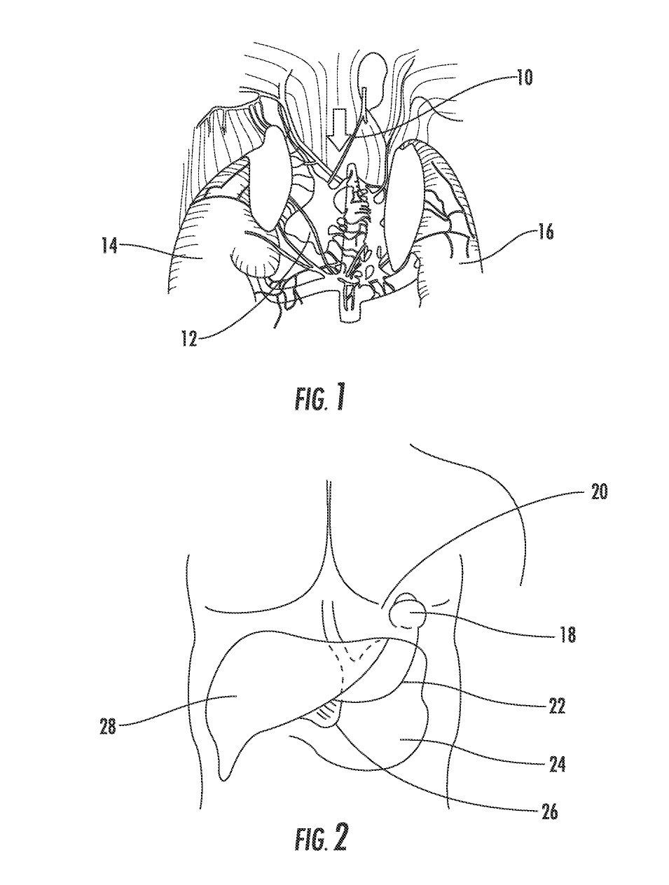

The anatomy of the dividing vagus beyond the oesophageal hiatus has been well studied by gastric surgeons from the era of selective vagotomy and is shown diagrammatically in FIG. 1. The celiac nerve is a stout and constant nerve that runs adjacent to the left gastric artery in the region of the lesser curve of the stomach (lesser omentum) (Anderson, 1983; FIG. 4). It provides the main connectivity between the posterior vagal trunk (lying subdiaphramatically at the oesopahgeal hiatus) and the celic plexus lying at the root of the foregut mesentery anterior to the aorta. As such it provides the main afferent and efferent vagal contributions to the foregut, and (via onward preaortic connections) to the midgut via the superior hypogastric plexus. The nerve, with an approximate diameter of 2 mm, is easily visible at open and laparoscopic surgery and can be readily dissected from the fat of the lesser omentum from either side (greater or lesser sac).

The described methods and systems include procedures which are useful via open or laparoscopic approaches depending on previous surgical history. The operation is optionally performed under general anaesthesia in a supine position. In the first instance this is optionally done by an open technique using an upper midline incision but a laparoscopic approach is also useful for suitable patients becoming akin to that for Gastric electrical stimulation.

During the procedure, the liver is optionally retracted, the lesser curvature of the stomach identified and the lesser omentum gently dissected to find the left gastric artery and coeliac nerve. A suitable length (approx. 2 cm) of nerve is optionally dissected free at 2 points to permit encirclage by the helical electrode retainers leaving the orientated flat electrode assembly lying along the nerve proximal to distal. These are optionally loosely sutured in place using 0.5 mm silastic nerve retractor sutures.

The lead is optionally secured to the adjacent corpus of the stomach using portholes on the wings and a non absorbable suture. The lead is optionally taken to the abdominal wall and thence tunnelled to a suitable location (dependent, for example, on body habitus—usually left upper quadrant) for the implantable stimulator. Antibiotic therapy is optioanlly administered for 48 h. The device is optionally switched on in hospital and programmed according to pre-defined protocols. Cardiac monitoring is optionally continued for the first 48 h.

Referring now to the drawings in general but particularly to FIG. 1, the celiac branch of the nervous system is identified at 10 extending downwardly towards the right celiac ganglion 12 between the right and left kidneys 14, 16 respectively. It is this nerve to which example methods as described herein are to be applied. The described methods include the application of an electrical stimulus or signal to the identified nerve and to do this one may optionally use an external or an implantable programmer (IPG).

FIG. 2 shows an implantable programmer 18 embedded in the left chest wall 20 with a supply lead 22 extending anterior to the stomach 24 to the lesser omentum 26. Also shown is the liver 28.

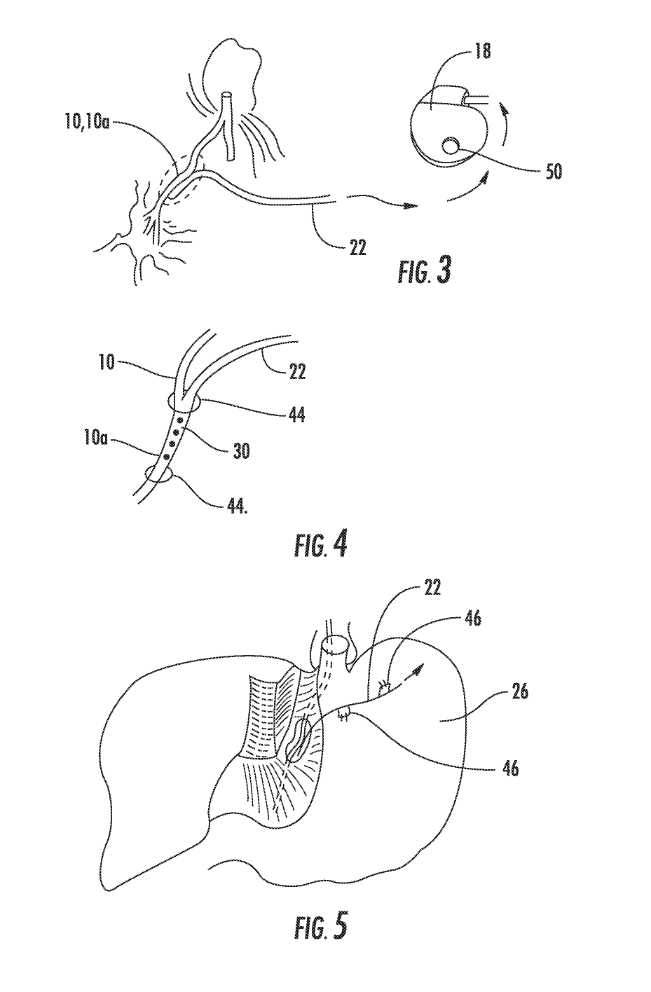

Referring now particularly to FIGS. 2 and 3 which show the installation of the electrode 30 in more detail, it is appreciated that the nerve branch is located and then separated for the application of the electrode 30. In this particular instance, the method comprises the steps of first identifying sub-diaphragmatic vagus nerve branches 10 and then applying the electrode 30 to said nerve branch. At present this is optionally done via open or laparoscopic approaches depending on previous surgical history and the operation is optionally performed under general anaesthesia in a supine position. When adopting an open technique an upper midline incision is optionally used, but a laparoscopic approach is also optionally used. The portion of the vagus nerve that is identified is optionally the celiac branch 10a or posterior vagal trunk of the sub-diaphragmatic vagus nerve and the electrode 30 is applied directly to said trunk or branch depending on ease of dissection 10a.

During the procedure, the liver is retracted, the lesser curvature of the stomach 24 identified and the lesser omentum gently dissected to find the left gastric artery and celiac nerve 10a (this can also be performed by a combined front and back approach, the latter via opening the lesser sac). A suitable length (approx. 2 cm) of nerve 10 is dissected free at two points to permit the application of electrode retainers 32 which are then applied leaving the orientated electrode 30 lying along the nerve 10 proximal to distal. These are optionally loosely sutured in place y sutures 34.

The lead 22 is optionally secured to the adjacent lesser curve or corpus of the stomach 24 using portholes 36 (FIG. 5) on the wings and a non absorbable suture 38. The lead is optionally taken to the abdominal 40 wall and thence tunnelled to a suitable location (dependent on body habitus—usually left upper quadrant) for the implantable stimulator 18. Antibiotic therapy is optionally administered for 48 h after the operation. The device is optionally switched on in hospital and programmed according to pre-defined protocols. Cardiac monitoring is optionally continued for the first 48 h post operation.

Subsequent to successful installation, stimulating the subject's vagus nerve branch by the application of electrical pulses through said electrode 30 to said nerve 10 in a given pattern and at a rate controlled by the controller 18 such as to relieve pain is commenced, as discussed in more detail later herein.

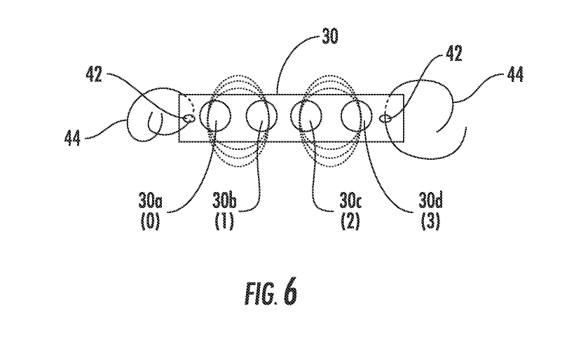

Referring now more particularly to FIG. 6, a single stimulation electrode 30 is provided with four linear electrodes 30a, 30b, 30c and 30d at a separation of approximately 5 mm (2 cm total). The electrodes in combination optionally deliver a directional uniphasic current in the safe therapeutic range (see below).

The electrode unit 30 is provided with a securing mechanism 40 in the form of, for example, eyelets 42 at each end which allow for the passage of a suture therethrough which is optionally secured to the nerve itself in a manner that allows at least one end to be relatively free floating relative to the nerve 10a. The arrangement optionally incorporate soft retaining helices 44 at approximately 10 mm separation (FIG. 3) that maintain adjacency without restricting axonal movement or blood supply (allowing for swelling of the nerve).

A single flexible narrow calibre insulated lead 22 is optionally provided from the IPG 18 to the electrode 30 and the lead is selected to have a narrow calibre and a high degree of flexibility to prevent adverse events such as internal herniation. The 22 lead is of sufficient length to reach the stimulator unit 18. The lead is optionally modified to incorporate a series of distal anchoring wings 46 (FIG. 5) to prevent adjacent lead movement by suturing through portholes 48 (not shown) on the wings 46 to the lesser curve of stomach 24.

The implantable programmable neurostimulator unit 18 is placed subcutaneously on the abdominal wall and is optionally provided with a default (efferent stimulation) mode, in which the stimulator generates a negative charge at a distal electrode 30a (cathode) and a positive charge at one of the more proximal anodes 30b, 30d, 30e. It is also possible, however, to set the base unit as the anode with one or both electrodes as cathodes, which produces a wider and more bidirectional stimulation field. The stimulator is optionally capable of a patient-activated higher amplitude and frequency cycle by means of override 50 that can be set independently of the background cycle parameters. This is optionally activated by the patient on an ‘on demand’ basis during exacerbations of abdominal pain using new generation of intelligent IPG's.

The stimulation parameters and safety issues are now discussed. Initial stimulation parameters are optionally derived from safe cervical vagal nerve stimulation e.g. 30 Hz frequency, 500 μs pulse width and 30-second On/5-minute OFF duty cycle with a current draw of 0.25-2.0 mA. The stimulation parameters are optionally varied, however, and the duty cycle can be altered within broad limits from a few seconds to several minutes. Such alterations optionally depend upon the patient response and the final treatment parameters are optionally determined accordingly. For the purpose of illustration provided below are example ranges of parameters below:

The electro pulses optionally comprise a directional uniphasic current. The current is optionally applied periodically. The current optionally comprises a current of between 0.25 mA and 2.0 mA. The current is optionally applied at a frequency of between 5 Hz and 50 Hz, for example, at substantially 30 Hz. The stimulation is optionally applied at intervals for a given duration and optionally has an applied on/off ration of between 1/10 and 1/1. The pulses are optionally applied for between 100 μs and 1000 μs, for example between 200 μs and 500 μs, and, for example, for 500 μs.

Well established left cervical vagus stimulation used for epilepsy (in the neck) delivers a mainly afferent stimulus centrally i.e. to the brainstem vagal nuclei with minor efferent effects limited to some hoarseness caused by recurrent laryngeal nerve stimulation. The two electrodes are thus set with the cathode proximally (the active charge in respect of nerve membrane depolarisation) and the relatively non-conducting (hyperpolarising) anode distally. The described systems and methods optionally utilizes uinphasic periodic stimulation, for example, (30 sec ON, 5 min OFF) with 30 Hz frequency, 500 μs pulse width and a current draw of 0.25-2.0 mA. Optionally, the described systems and methods utilizes a four electrode arrangement as illustrated in the figures attached hereto and to which the process can be applied by applying the cathode and electrode to any one or other of the electrodes 30. These electrodes are more commonly referred to as 0, 1, 2 and 3. By varying the electrodes used, the distance over which the current path is generated and the location is optionally altered to enlarge or reduce the area of stimulation. Additionally, the direction of current flow is optionally varied by altering the position of the anode and cathode. Such variation is optionally implemented in response to patient demand for alteration of the treatment and may be implemented by the patient himself.

Neuromodulation of the vagus below the diaphragm has previously been directed at producing a conduction (anodal) block to, broadly speaking, impede GI motility and thus treat obesity. The firing frequencies of small unmyelinated afferents that constitute the vagus nerve (up to 500 Hz) cannot be usefully blocked at safe frequencies and amplitudes of chronic stimulation (5,000 Hz, 6 mA) chosen in obesity. Indeed, unpublished observations suggest that the high frequency blocking stimulation has been associated with adverse events in obese patients. Instead, the provided systems and methods use lower frequency and amplitude pulses similar to tried and tested cervical VNS for epilepsy. Stimulation is optionally directed in a descending direction by a distal cathode directly to stimulate the coeliac plexus rather than in an ascending direction to act upon the CNS. Adhering to cervical VNS stimulation parameters has major advantages regarding safety issues in any required pilot studies. As with cervical VNS, the frequency, pulse width, and cycling parameters are optionally programmable such that alternative pulse widths e.g. 250 μs are optionally used. The distal position of the electrodes in relation to the thoracic vagal outflow to the heart has the added advantage of making cardiac side effects unlikely, although these should be closely monitored.

The disclosed systems and methods have no direct competition in the treatment of patients with chronic pain using electrical neurostimulation techniques. Existing technologies e.g. Interstim® and Enterra® and ongoing attempts at direct stimulation of the intestinal wall (Xu et al.,2011) are directed at modifying motility (and do not necessarily address symptoms—noting that there are many treatments that accelerate transit but which have little sustained effect in terms of patient symptomatic benefit). Although the primary aim is to address visceral pain, there is some evidence that stimulation of the vagus also has secondary beneficial effects on (1) dysmotility, (2) inflammation, (3) nausea and vomiting, (4) depression and anxiety and (5) somatic pain syndromes e.g. fybomyalgia, all of which frequently co-exist to some degree in such patients. The disclosed systems and methods are optionally used in other disorders characterized by dysregulated visceral immunity e.g: Inflammatory bowel disease (particularly recurrent multilevel Crohn's stricturing / fistulation), Prevention of rejection after visceral transplantation and Abdominal polytrauma.

A number of embodiments of the invention have been described. Nevertheless, it will be understood that various modifications may be made without departing from the spirit and scope of the invention. Accordingly, other embodiments are within the scope of the following claims.

Publications cited herein and the materials for which they are cited are hereby specifically incorporated by reference in their entireties.

What is claimed is:

1. A method of abdominal pain relief in a subject comprising the steps of:
a. identifying a sub-diaphragmatic vagus nerve branch;
b. applying an electrode to said nerve branch;
c. stimulating the subject's vagus nerve branch by the application of electrical pulses through said electrode to said nerve in a given pattern and at a rate controlled by a controller such as to relieve pain.

2. The method of claim 1, wherein the branch of the vagus nerve that is identified is the celiac branch or posterior vagal trunk of the sub-diaphragmatic vagus nerve and the electrode is applied directly to said branch.

3. The method of claim 1, wherein the electrical pulses comprise a directional uniphasic current.

4. The method of claim 3, wherein the current is applied periodically.

5. The method of claim 3, wherein the current comprises a current of between 0.25 mA and 2.0 mA.

6. The method of claim 3, wherein the current is applied at a frequency of between 5 Hz and 50 Hz.

7. The method of claim 6, wherein the current is applied at substantially 30 Hz.

8. The method of claim 4, wherein the stimulation is applied at intervals for a given duration.

9. The method of claim 7, wherein the stimulation is applied in an on/off ration of between 1/10 and 1/1.

10. The method of claim 1, wherein the pulses are applied for between 100 μs and 1000 μs.

11. The method of claim 10, wherein the pulses are applied for between 200 μs and 500 μs.

12. The method of claim 1, wherein the pulses are applied for 500 μs.

13. The method of claim 1, wherein the stimulation is applied via an electrode having a plurality of electrode plates positioned along and alongside the nerve and electrical pulses are applied between different pairs thereof in accordance with a pre-defined protocol.

14. The method of claim 13, further comprising securing the electrode to said nerve.

15. The method of claim 10, further comprising securing an electrical lead used for the supply of electricity to said electrode to the anterior wall of the stomach.

16. The method of claim 12, wherein said leads are secured by means of a slide coupling allowing the lead to slide within the coupling, thereby to accommodate movement.
